(19) United States
(12) Patent Application Publication
(10) Pub. No.: US 2023/0210978 A1
(43) Pub. Date: Jul. 6, 2023

(54) A UNIVERSAL ADOPTIVE IMMUNOTHERAPY APPROACH TO TREAT COVID-19 AND FUTURE EMERGING INFECTIOUS DISEASES

(71) Applicants: Francois BINETTE, Carlsbad, CA (US); Brian M. CULLEY, Carlsbad, CA (US); Rami SKALITER, Carlsbad, CA (US); LINEAGE CELL THERAPEUTICS, INC., Carlsbad, CA (US)

(72) Inventors: Francois BINETTE, Napa, CA (US); Brian M. CULLEY, Carlsbad, CA (US); Rami SKALITER, Ness Ziona (IL)

(21) Appl. No.: 17/995,008

(22) PCT Filed: Apr. 2, 2021
(86) PCT No.: PCT/US21/25638

§ 371 Date: Sep. 29, 2022

Related U.S. Application Data
(60) Provisional application No. 63/063,944, filed on Aug. 10, 2020.
(60) Provisional application No. 63/004,408, filed on Apr. 2, 2020.

(51) Int. Cl. A61K 39/215; C12N 5/0784; A61P 31/14

(52) U.S. Cl. CPC A61K 39/215; C12N 5/0639; A61P 31/14; C12N 2770/20034; A61K 2039/5154

(57) ABSTRACT

Provided herein are methods and compositions for engineering antigen-presenting cells (APCs), such as dendritic cells (DCs), macrophages, and B-Cells, that are modified to express viral antigens. With this approach, large cell banks can be created that can be rapidly modified and deployed to fight various types of infectious diseases such as those associated with coronaviruses and other viral infections.

### CROSS-REFERENCES TO RELATED APPLICATIONS

This application claims the benefit of U.S. Provisional Application No. 63/004,408, filed Apr. 2, 2020, and U.S. Provisional Application No. 63/063,944, filed Aug. 10, 2020, which are incorporated herein by reference in their entireties and for all purposes.

### BACKGROUND

Coronaviruses belong to the family Coronaviridae and are enveloped, positive-sense, single-stranded RNA viruses. The coronavirus genome is approximately 31 kb in size, making these viruses the largest known RNA viruses yet identified. Coronaviruses infect a variety of hosts including humans and several other vertebrates. Coronaviruses are associated with several respiratory and intestinal tract infections. Respiratory coronaviruses have long been recognized as significant pathogens in domestic and companion animals and as the cause of upper respiratory tract infections in humans. Thus, several human coronaviruses (HCoVs) are the etiological agents for mild respiratory illness, including the common cold and croup (e.g.: HCoV-229E, HCoV-OC43, HCoV-NL63 and HCoV-HKU), and now SARS-CoV-2, which causes COVID-19.

The current COVID-19 pandemic is a clear demonstration of the enormous risks that such emerging infectious diseases might impose on global health and the worldwide economy. From a therapeutic development perspective, emerging threats such as SARS-CoV-2 are intrinsically difficult to address in a timely manner because of the incredible pace of propagation, in part because of the general connectivity of people traveling and interacting on a global basis. As such, therapeutic readiness is focused on platform development which can be readily and rapidly adapted to address specific pathogens, or their clinical manifestation in people. Moreover, the rapid evolution of complex and poorly understood virulence factors challenges the ability of those affected to mount an effective immune response to fight and/or develop a memory immunity to block recurring infections. There is a need to 1) address immediate impact of COVID-19 to curb the burden on healthcare systems, and 2) stockpile large arsenals of complementary tools to combat future emerging threats. The dendritic cell (DC) vaccine platform described herein may address both by mobilizing immune systems to specifically target SARS-CoV-2 viral particles and infected cells, while large cell banks can be created to adapt quickly to future infectious diseases.

### BRIEF SUMMARY OF THE INVENTION

In one aspect, the present disclosure provides a method of making a composition for treating an infectious disease, said method comprising obtaining a cell line of human pluripotent stem (hPS) cells; differentiating said hPS cells into a population of mature antigen-presenting cells, wherein said antigen expressing cells express an antigen of a Coronaviridae virus; genetically altering said hPS cells before or after they are differentiated so that they express a protein comprising one or more immunogenic epitopes of said Coronaviridae virus; and formulating said differentiated cells to provide a composition for administration to a human subject.

In some embodiments, the Coronaviridae virus is SARS-CoV-2.

In some embodiments, the antigen-presenting cells are selected from the group consisting of dendritic cells, macrophages, B cells, and a combination thereof.

In some embodiments, the composition is administered to a human for treating a disease caused by a Coronaviridae virus infection.

In some embodiments, the composition is administered to a human for preventing a disease caused by a Coronaviridae virus infection.

In some embodiments, the composition is a vaccine.

In some embodiments, the Coronaviridae antigen comprises the Coronaviridae spike protein. In some embodiments, the Coronaviridae antigen comprises the full-length spike protein. In some embodiments, the Coronaviridae antigen comprises the spike protein receptor-binding domain (RBD). In some embodiments, the Coronaviridae antigen comprises a 209 amino acid sequence of the RBD. In some embodiments, the Coronaviridae antigen comprises the spike protein S1 domain, S2 domain, or both. In some embodiments, the Coronaviridae antigen comprises the nucleocapsid protein. In some embodiments, the Coronaviridae antigen comprises the membrane protein.

In some embodiments, genetically altering said hPS cells comprises transfecting the cells with a polynucleotide encoding a Coronaviridae virus antigen and the cytoplasmic tail of lysosomal associated membrane protein 1 (LAMP-1). In some embodiments, the polynucleotide further comprises a poly(A) tail and a gene which encodes heat shock protein 96 (HSP96).

In another aspect, the present disclosure provides a composition according to any one of the methods described herein.

In some embodiments, the Coronaviridae virus is SARS-CoV-2.

In some embodiments, the antigen-presenting cells are selected from the group consisting of dendritic cells, macrophages, B cells, and a combination thereof. In some embodiments, the antigen-presenting cells are dendritic cells.

In some embodiments, the composition is administered to a human for treating a disease caused by a Coronaviridae virus infection.

In some embodiments, the composition is administered to a human for preventing a disease caused by a Coronaviridae virus infection.

In some embodiments, the Coronaviridae antigen comprises the Coronaviridae spike protein. In some embodiments, the Coronaviridae antigen comprises the full-length spike protein. In some embodiments, the Coronaviridae antigen comprises the spike protein receptor-binding domain (RBD). In some embodiments, the Coronaviridae antigen comprises a 209 amino acid sequence of the RBD. In some embodiments, the Coronaviridae antigen comprises the spike protein S1 domain, S2 domain, or both. In some embodiments, the Coronaviridae antigen comprises the nucleocapsid protein. In some embodiments, the Coronaviridae antigen comprises the membrane protein.

In some embodiments, genetically altering said hPS cells comprises transfecting the cells with a polynucleotide encoding a Coronaviridae virus antigen and the cytoplasmic tail of lysosomal associated membrane protein 1 (LAMP-1). In some embodiments, the polynucleotide further comprises a poly(A) tail and a gene which encodes heat shock protein 96 (HSP96).

In some embodiments, the composition is a vaccine.

In yet another aspect, the present disclosure provides a composition comprising dendritic cells expressing a Coronaviridae antigen.

In some embodiments, the Coronaviridae antigen comprises the Coronaviridae spike protein. In some embodiments, the Coronaviridae antigen comprises the full-length spike protein. In some embodiments, the Coronaviridae antigen comprises the spike protein receptor-binding domain (RBD). In some embodiments, the Coronaviridae antigen comprises a 209 amino acid sequence of the RBD. In some embodiments, the Coronaviridae antigen comprises the spike protein S1 domain, S2 domain, or both. In some embodiments, the Coronaviridae antigen comprises the Coronaviridae nucleocapsid protein. In some embodiments, the Coronaviridae antigen comprises the Coronaviridae membrane protein.

In some embodiments, genetically altering said hPS cells comprises transfecting the cells with a polynucleotide encoding a Coronaviridae virus antigen and the cytoplasmic tail of lysosomal associated membrane protein 1 (LAMP-1). In some embodiments, the polynucleotide further comprises a poly(A) tail and a gene which encodes heat shock protein 96 (HSP96).

### BRIEF DESCRIPTION OF THE DRAWINGS

FIG. 1—schematic describing the Dendritic Cell Infectious Diseases Program.

FIG. 2—graph describing the features and results derived from dendritic cell vaccines.

FIG. 3—shows relevant non-clinical data obtained with dendritic cell vaccines.

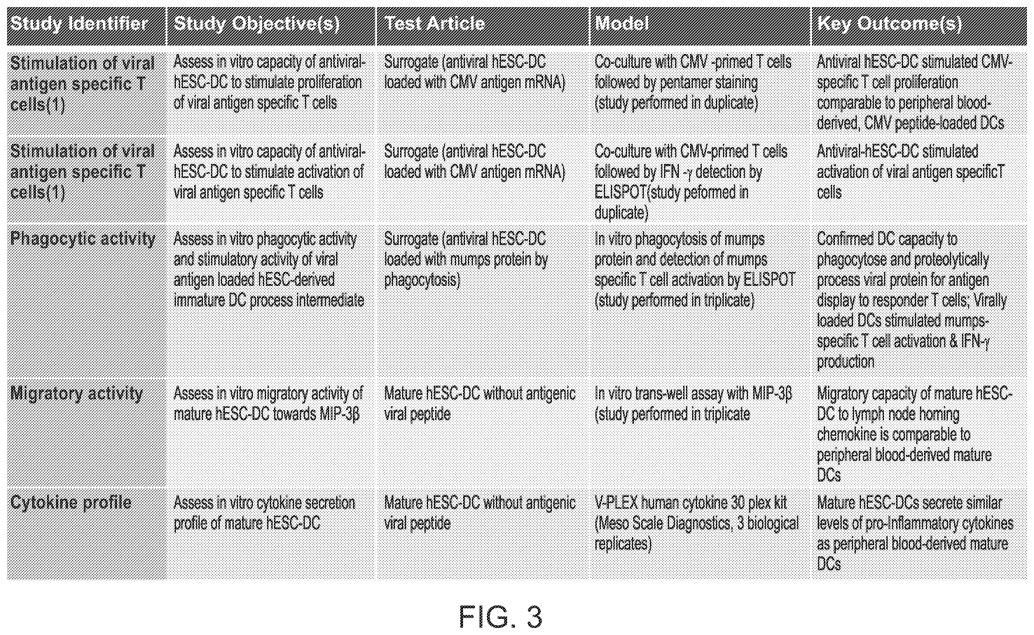

FIG. 4—provides approaches for scaling up production of vaccines.

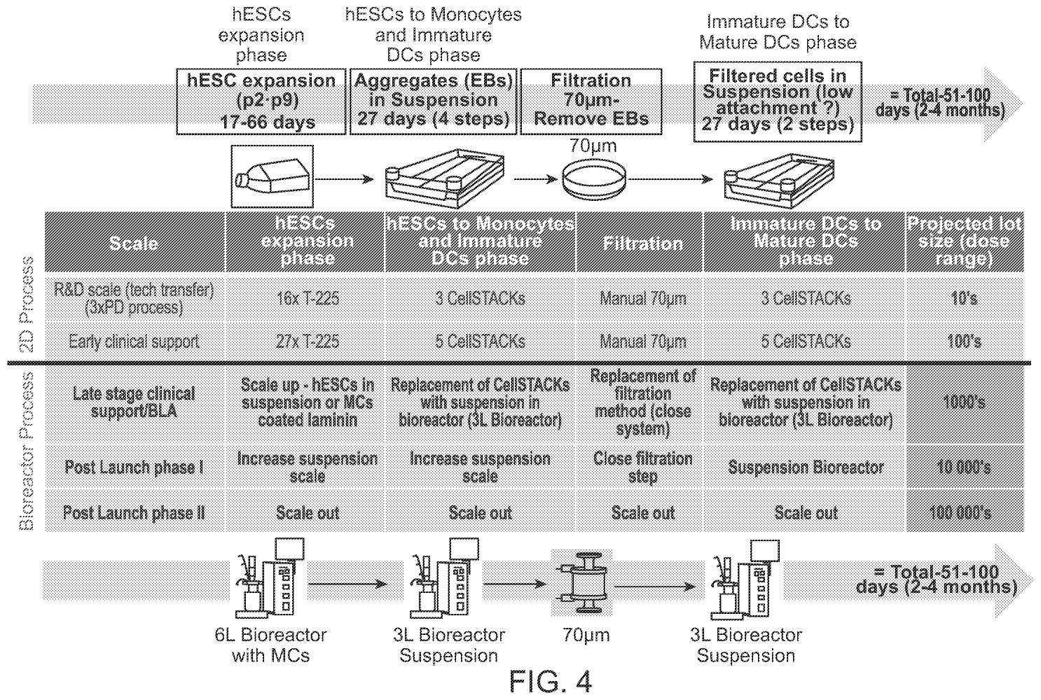

FIG. 5—shows flow cytometry graphs for the various groups studied in Example 3.

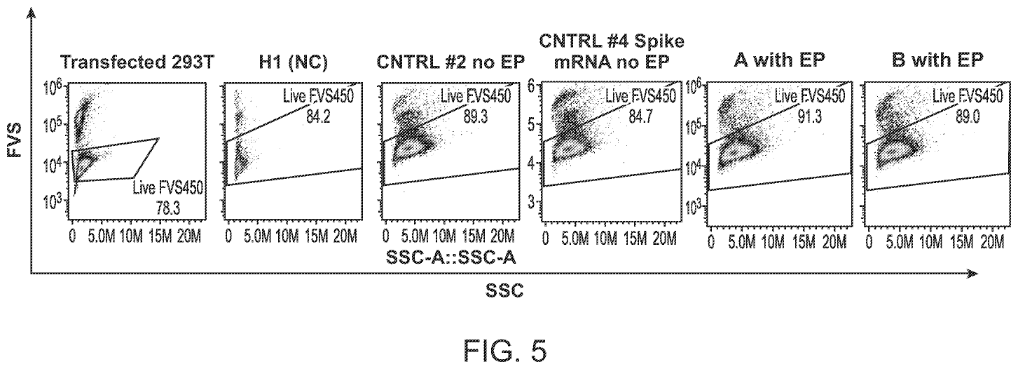

FIG. 6—shows flow cytometry data for efficacy of incorporation of spike antigen into transfected cells by electroporation.

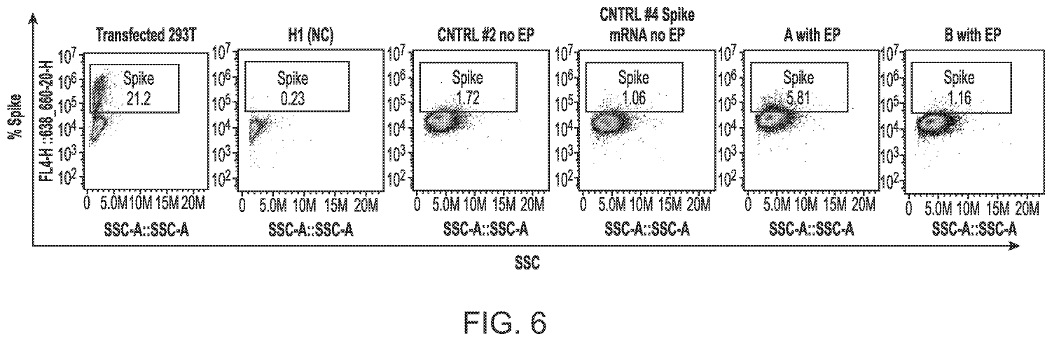

FIG. 7—shows flow cytometry data for % incorporation of spike antigen versus expression of CD86.

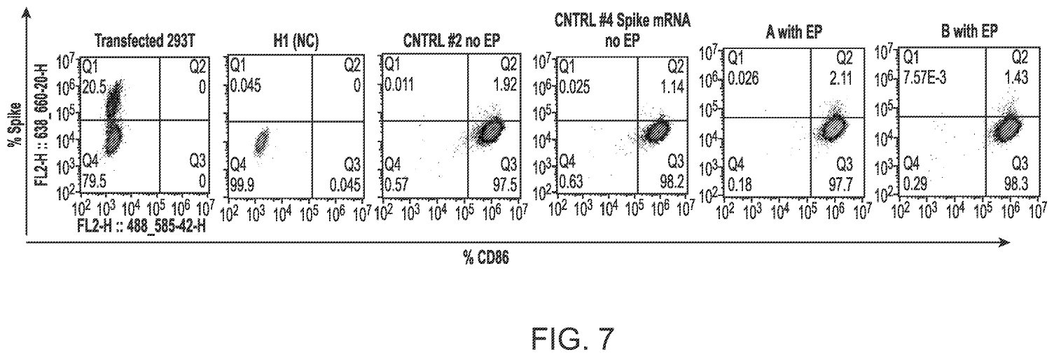

FIG. 8—shows flow cytometry data for % HLA-DR expression versus % expression of CD83.

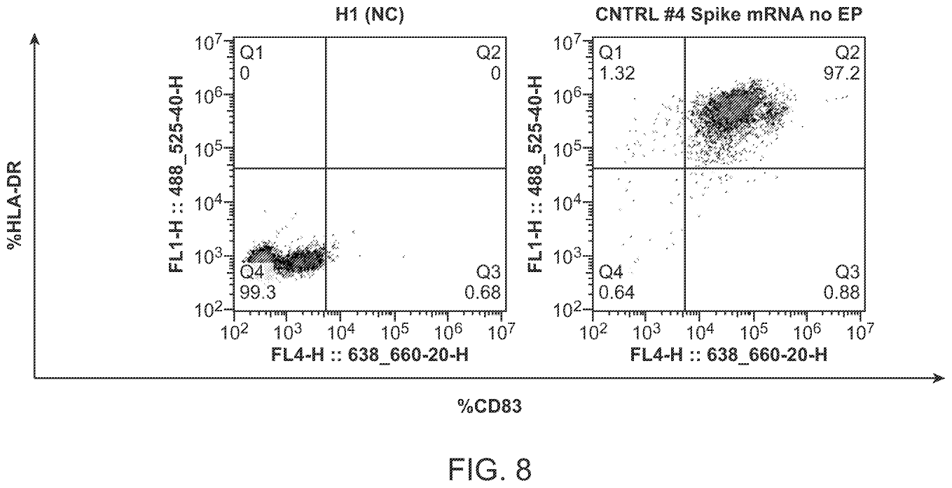

FIG. 9—restriction map showing the pKAN-SARS-CoV-2 (COVID 19)-LAMP1 construct with a Kanamycin resistance gene.

FIG. 10—schematic diagram of the plasmid used to produce the SARS-CoV-2 (COVID 19) spike protein/LAMP-1 mRNA.

FIG. 11—denaturing Agarose gel showing size of the mRNA synthesized from the SARS-CoV-2 (COVID 19)-LAMP1 plasmid construct. FlashGel™ RNA marker (ladder) was loaded on lane 1 and lane 12. FlashGel™ RNA marker consists of RNA transcripts 0.5 kb-9 kb. Lanes 2-11 show mRNA loaded at different concentrations as shown in the right-hand side of the image. Lane 13 is a no template control (negative control).

FIG. 12—schematic diagram of the SARS-CoV-2S LAMP-1 mRNA construct.

### DETAILED DESCRIPTION

### I. Definitions

Cell culture, as used herein, refers to a plurality of cells grown in vitro over time. The cell culture may originate from a plurality of hPS cells or from a single hPS cell and may include all of the progeny of the originating cell or cells, regardless of 1) the number of passages or divisions the cell culture undergoes over the lifetime of the culture; and 2) any changes in phenotype to one or more cells within the culture over the lifetime of the culture (e.g. resulting from differentiation of one or more hPS cells in the culture). Thus, as used herein, a cell culture begins with the initial seeding of one or more suitable vessels with at least one hPS cell and ends when the last surviving progeny of the original founder(s) is harvested or dies. Seeding of one or more additional culture vessels with progeny of the original founder cells is also considered to be a part of the original cell culture.

Coronaviruses belong to the family Coronaviridae and are enveloped, positive-sense, single-stranded RNA viruses. The coronavirus genome is approximately 31 kb in size, making these viruses the largest known RNA viruses yet identified. Coronaviruses infect a variety of hosts including humans and several other vertebrates. Coronaviruses are associated with several respiratory and intestinal tract infections. Respiratory coronaviruses have long been recognized as significant pathogens in domestic and companion animals and as the cause of upper respiratory tract infections in humans. Thus, several human coronaviruses (HCoVs) are the etiological agents for mild respiratory illness, including the common cold and croup (e.g.: HCoV-229E, HCoV-OC43, HCoV-NL63 and HCoV-HKU). Human coronaviruses such as SARS-CoV and MERS-CoV are also associated with severe respiratory illness. Coronaviruses that induce respiratory tract disease in other vertebrate animals include mouse hepatitis virus-1 (MHV-1) a natural mouse pathogen, infectious bronchitis virus (IBV) in chickens and other avian species, bovine coronavirus (BCoV) in cows and other ruminants, porcine respiratory syndrome virus (PRCV) in pigs and canine respiratory coronavirus (CRCoV) in dogs to name a few. (See, for example, Refs. 1-11).

Dendritic cells (DCs) are professional antigen-presenting cells (also known as accessory cells) of the mammalian immune system. Their main function is to process antigen material and present it on the cell surface to the T cells of the immune system. They act as messengers between the innate and the adaptive immune systems.

The terms “treatment” and “treating” as used herein refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In one aspect, the compounds described herein can be administered to a subject comprising a human or an animal including, but not limited to, a mouse, dog, cat, horse, bovine or ovine and the like, that is in need of alleviation or amelioration from a recognized medical condition. In specific embodiments, the subject is human.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the disclosed compounds.

The term “preventing” as used herein refers to administering a compound prior to the onset of clinical symptoms of a disease or conditions so as to prevent a physical manifestation of aberrations associated with the disease or condition. In the context of SARS-CoV-2 infection, the term “preventing” refers to administering a compound prior to the onset of clinical symptoms of SARS-CoV-2 virus infection so as to prevent a physical manifestation of aberrations associated with SARS-CoV-2 virus infection.

As used herein, “feeder cells” refers to non-hPS cells that are co-cultured with hPS cells and provide support for the hPS cells. Support may include facilitating the growth and maintenance of the hPS cell culture by providing the hPS cell culture with one or more cell factors such that the hPS cells are maintained in a substantially undifferentiated state. Feeder cells may either have a different genome than the hPS cells or the same genome as the hPS cells and may originate from a non-primate species, such as mouse, or may be of primate origin, e.g., human. Examples of feeder cells may include cells having the phenotype of connective tissue such as murine fibroblast cells, human fibroblasts.

As used herein, “feeder-free” refers to a condition where the referenced composition contains no added feeder cells. To clarify, the term feeder-free encompasses, inter alia, situations where primate pluripotent stem cells are passaged from a culture which may comprise some feeders into a culture without added feeders even if some of the feeders from the first culture are present in the second culture.

“Serum free”, as used herein, refers tissue culture growth conditions that have no added animal serum such fetal bovine serum, calf serum, horse serum, and no added commercially available serum replacement supplements such as B-27. Serum free includes, for example, media which may comprise human albumin, human transferrin and recombinant human insulin.

As used herein, “electroporation” refers to a method for permeabilizing cell membranes by generating membrane pores with electrical stimulation. The applications of electroporation include, but are not limited to, the delivery of DNA, RNA, siRNA, peptides, proteins, antibodies, drugs or other substances to a variety of cells such as mammalian cells, plant cells, yeasts, other eukaryotic cells, bacteria, other microorganisms, and cells from human patients.

### II. Methods

In an aspect, provided herein are methods of making a composition for treating an infectious disease, the method including: a) obtaining a cell line of human pluripotent stem (hPS) cells; b) differentiating said hPS cells into a population of mature antigen-presenting cells, wherein said antigen expressing cells express an antigen of an infectious disease; c) genetically altering said hPS cells before or after they are differentiated so that they express a protein comprising one or more immunogenic epitopes of said infectious disease; and d) formulating said differentiated cells to provide a composition for administration to a human subject.

Methods of Differentiating

Pluripotent stem cells have the ability to both proliferate continuously in culture and, under appropriate growth conditions, differentiate into lineage restricted cell types representative of all three primary germ layers: endoderm, mesoderm and ectoderm (U.S. Pat. Nos. 5,843,780; 6,200,806; 7,029,913; Shamblott et al., (1998) Proc. Natl. Acad. Sci. USA 95:13726; Takahashi et al., (2007) Cell 131(5):861; Yu et al., (2007) Science 318:5858).

A pluripotent stem cell (PS) will, under appropriate growth conditions, be able to form at least one cell type from each of the three primary germ layers: mesoderm, endoderm and ectoderm. The PS cells may originate from pre-embryonic, embryonic or fetal tissue or mature differentiated cells. Alternatively, an established PS cell line may be a suitable source of cells for practicing the invention. Typically, the hPS cells are not derived from a malignant source. hPS cells will form teratomas when implanted in an immuno-deficient mouse, e.g. a SCID mouse.

Prototype “human Pluripotent Stem cells” (hPS cells) are pluripotent cells derived from pre-embryonic, embryonic, or fetal tissue at any time after fertilization, and have the characteristic of being capable under appropriate conditions of producing progeny of several different cell types that are derivatives of all of the three germinal layers (endoderm, mesoderm, and ectoderm), according to a standard art-accepted test, such as the ability to form a teratoma in 8-12 week old SCID mice. Unless otherwise specified, hPS cells are not derived from a cancer cell or other malignant source. It is desirable (but not always necessary) that the cells be euploid.

Exemplary are embryonic stem cells and embryonic germ cells used as existing cell lines or established from primary embryonic tissue of human origin. This invention can also be practiced using pluripotent cells obtained from primary embryonic tissue, without first establishing an undifferentiated cell line.

hPS cells can be propagated continuously in culture, using culture conditions that promote proliferation while inhibiting differentiation. Traditionally, ES cells are cultured on a layer of feeder cells, typically fibroblasts derived from embryonic or fetal tissue (Thomson et al., Science 282:1145, 1998).

However, hPS cells can be maintained in an undifferentiated state even without feeder cells. The environment for feeder-free cultures includes a suitable culture substrate, such as ECM-based hydrogel, such as solubilized basement membrane preparation extracted from the Engelbreth-Holm-Swarm (EHS) mouse sarcoma (e.g., MATRIGEL®), or laminin. The cultures are supported by a nutrient medium containing factors that promote proliferation of the cells without differentiation (WO 99/20741). Such factors may be introduced into the medium by culturing the medium with cells secreting such factors, such as irradiated primary mouse embryonic fibroblasts, telomerized mouse fibroblasts, or fibroblast-like cells derived from hPS cells (U.S. Pat. No. 6,642,048). Medium can be conditioned by plating the feeders in a serum free medium such as Knock-Out DMEM (Gibco), supplemented with serum replacement ranging from 10-30%, preferably 20% (US 2002/0076747 A1, Life Technologies Inc.) and 4 ng/mL bFGF. Medium that has been conditioned for 1-2 days is supplemented with further bFGF, and used to support hPS cell culture for 1-2 days (WO 01/51616; Xu et al., Nat. Biotechnol. 19:971, 2001).

Some embodiments of the invention provide for maturing immature DC (imDC) to mature DC (mDC) by contacting the imDC with a suitable maturation cocktail comprising a plurality of exogenous cytokines. The maturation cocktail may comprise GM-CSF. Examples of suitable maturation cocktails include any of the following: a) GM-CSF, TNFa, IL-1p, IFNy, and PGE2; b) GM-CSF, TNFa, IL-Ip, IFNy, PGE2 and CD40L; c) GM-CSF, TNFa, IL-Ip, IFNy, PGE2, POLY I:C, and IFNa; d) GM-CSF, TNFa, IL-1p, IFNy, POLY I:C, and IFNa; e) GM-CSF, TNFa, IL-Ip, IFNy, POLY I:C, IFNa, and CD40L; f) TNFa, IL-1p, PGE2 and IL-6; g) GM-CSF, IL-1p, PGE2, and, IFNy; h) GM-CSF, TNFa, PGE2, and, IFNy; i) GM-CSF, IL-1 p, IFNy and CD40L. In some embodiments ligands to one or more cytokine receptors may be used in place of, and/or in addition to the cytokine. Other methods, known in the art, may be used to mature imDC to mDC. Examples include contacting imDC with lipopolysaccharide (LPS), contacting the imDC with CpG containing oligonucleotides, injecting the imDC into an area of inflammation within a subject.

The imDC may be cultured in the presence of the maturation cocktail, for at least about 12-15 hours, for at least about 1 day, for at least about 2 days, or for at least about 3 days to produce mDC. In some embodiments the imDC may be cultured in the presence of the maturation cocktail for about 24 hours to produce mDC. In other embodiments the imDC may be cultured in the presence of the maturation cocktail for about 48 hours to produce mDC.

mDC may express one or more markers such as CD83, CD86, MHCI and MHCII, but not CD 14 and may have functional properties similar to mature DC that are differentiated in vivo. Functional properties may include the capability to process and present antigen to an immunologically competent cell. Processing and presenting antigen may include for example the proteolysis of a target protein, as well as the expression and processing of a nucleic acid encoding a target antigen. The mDC may also have the ability to migrate within peripheral and lymphoid tissue. Thus mDC differentiated from hPS cells according to the invention may be induced to migrate in response to an appropriate stimulus such as MIP3p. The mDC may secrete one or more cytokines such as one or more pro-inflammatory cytokines. Exemplary cytokines secreted by DC according to the invention may include IL-12, IL-10 and IL-6.

In specific embodiments, the cells express one or more markers of CD86, CD83, or MHCII.

Various embodiments of the invention described herein provide methods of differentiating hPS cells into DCs. It is contemplated that the methods may further comprise mitotically inactivating various types of cells including unwanted hPS cells in a differentiated population as well as cells made according the methods described infra (e.g. any hematopoietic lineage cells, including mDC and imDC). Thus some embodiments of the invention may comprise contacting the DC cells with a protein or peptide antigen or a nucleic acid encoding an antigen and contacting the DC e.g. an mDC, with a radiation source or a chemical agent suitable for inhibiting cell division. Exposure of the mDC to a radiation source or the chemical agent may be desirable where the mDC are contained in a population of cells comprising at least one hPS cell. Irradiating the cells or treating the cells with the chemical agent will inhibit cell division, while maintaining functionality of the mDC. Moreover, treating the cells with a radiation source or a chemical agent may minimize any undesirable effects stemming from the presence of hPS cells in the population.

In some embodiment the invention provides a method of differentiating hPS cells into mesoderm comprising contacting the hPS cells with a differentiation cocktail comprising a plurality of exogenous cytokines such as BMP-4, VEGF, SCF and optionally GM-CSF and culturing the cells for at least a day thereby differentiating hPS cells into mesoderm. In some embodiments the cells may be cultured for at least about 2 days, at least about 3 days, at least about 4 days, at least about 5 days with the differentiation cocktail thereby differentiating the hPS cells into mesoderm. In certain embodiments the hPS cells may be cultured with a differentiation cocktail for about 5 days in order to differentiate the hPS cells into mesoderm. In some embodiments the differentiation cocktail may optionally further comprise one or more of the following: FLT3L, TPO, IL-4 and IL-3. The mesoderm cells may express one or more factors or markers expressed by mesoderm cells. For example increased expression of the mesoderm associated transcription factor, Brachyury, along with the decreased expression of hPS associated transcription factor Oct4 and Tra-160 may be indicative of the differentiation of hPS cells to mesoderm cells. Allowing the culture to continue to grow in the presence of the differentiation cocktail may facilitate further differentiation of the mesoderm cells, e.g. into cells of hematopoietic lineage.

Thus in some embodiments the cell culture may be grown in the presence of the differentiation cocktail for a suitable length of time to differentiate the cells beyond mesoderm cells and into other hematopoietic lineage cells.

For example the cells may be grown at least about 6 days, at least about 7 days, at least about 8 days, at least about 9 days, at least about 10 days with the differentiation cocktail described herein thereby differentiating the hPS cells into hematopoietic stem cells. The cells may express one or more markers expressed by hematopoietic stem cells. Suitable markers may include CD45, CD34, and HoxB4. In yet further embodiments the cells may be grown at least about 22 days, at least about 23 days, at least about 24 days, at least about 25 days, at least about 26 days, at least about 27 days, at least about 28 days with the differentiation cocktail described herein thereby differentiating the hPS cells into monocytes. The cells may express one or more markers expressed by monocytes. Suitable markers may include CD 14, CD45 and CD 11 c. In still further embodiments the cells may be grown at least about 20 days, at least about 23, at least about 25 days, at least about 30 days, at least about 31 days, at least about 32 days, at least about 33 days, with the differentiation cocktail described herein thereby differentiating the hPS cells into imDC.

In certain embodiments the invention provides a method of differentiating hPS cells in hematopoietic lineage cells comprising contacting the hPS cells with one or more differentiation cocktails such that the hPS cells differentiate into one or more hematopoietic lineage cell types. The method may be comprised of multiple steps wherein one or more of the steps results in the differentiation of intermediate cell types of hematopoietic lineage. The invention contemplates not only the execution of all of the steps set forth below, but also the execution of one or more individual steps in order to attain a desired intermediate or precursor cell type of hematopoietic lineage.

In further embodiments the mesoderm cells from above may be contacted with a second differentiation cocktail comprising VEGF, SCF, GM-CSF thereby differentiating the mesoderm cells into hematopoietic stem cells. The cells may be cultured with this differentiation cocktail for about 1-5 days. In further embodiments hematopoietic the stem cell may be further differentiated into a common myeloid progenitor (CMP) cell by contacting the hematopoietic stem cell with a differentiation cocktail comprising GMCSF. For this step the differentiation cocktail may further comprise SCF. The cells may be cultured with this differentiation cocktail for about 1-10 days. In some embodiments the CMP may be further differentiated into a common granulocytic/monocytic progenitor (GMP) cell by contacting the CMP with a third differentiation cocktail comprising GMCSF.

The cells may be cultured with this differentiation cocktail for about 1-5 days. In further embodiments the GMP may be further differentiated into monocytes by contacting the GMP with a differentiation cocktail comprising GM-CSF. The cells may be cultured with this differentiation cocktail for about 1-10 days. In still further embodiments the monocytes may be further differentiated into imDC by contacting the monocytes with a differentiation cocktail comprising GM-CSF and IL-4. The cells may be cultured with this differentiation cocktail for about 1-5 days. In yet further embodiments the imDC may be matured into mDC by contacting the imDC with any of the maturation cocktails described infra. The cells may be cultured with the maturation cocktail from about 12-72 hours. In some embodiments the cells may be cultured with the maturation cocktail for about 24 hours. In other embodiments the cells may be cultured with the maturation cocktail for about 48 hours. The time may be any value or subrange within the recited ranges, including endpoints.

In still other embodiments the invention provides a method of differentiating hPS cells into imDC comprising contacting the hPS cells with a differentiation cocktail comprising the following: 1) BMP-4 ranging from about 10 ng/ml to about 75 ng/ml; and 2) GM-CSF ranging from about 25 ng/ml to about 75 ng/ml. The concentration of each may be any value or subrange within the recited ranges, including endpoints.

In still other embodiments the invention provides a method of differentiating hPS cells into imDC comprising contacting the hPS cells with a differentiation cocktail comprising the following: 1) BMP-4 ranging from about 10 ng/ml to about 75 ng/ml; 2) VEGF ranging from about 25 ng/ml to about 75 ng/ml; 3) SCF ranging from about 5 ng/ml to about 50 ng/ml; and 4) GM-CSF ranging from about 25 ng/ml to about 75 ng/ml. The concentration of each may be any value or subrange within the recited ranges, including endpoints.

In a further embodiment the invention provides a method of enriching a myeloid progenitor cell population comprising isolating a CD45+ Hi population from a cell culture comprising a CD45+ Hi cell population and a CD45+ low cell population. In a further embodiment the invention provides a method of isolating a granulocyte progenitor cell comprising isolating a CD45+ low population from a cell culture comprising a CD45+ Hi cell population and a CD45+ low cell population. High and low are relative terms. Thus a CD45+ low cell population may refer to a cells having CD45 expression about 1-2 orders of magnitude above background, while the CD45+ Hi cells may refer to cells having CD45 expression greater than 2 orders of magnitude above background as measured using any assay know in the art, e.g. immunofluorescence as measured using a fluorescence detector, e.g. Fluorescent Activated Cell Sorter (FACS). Isolating the target cell population may be done using any means known in the art. For example, the cell populations may be isolated using a commercially available (FACS). In some embodiments the cells may be isolated based on fluorescent intensity of a marker stained with a labeled ligand. The labeled ligand may attach directly to the cell or indirectly to the cell by virtue of another ligand attached to the cell by the human hand. The cell populations may be isolated based on size and density based on forward and side scatter on a cell sorter. As an example CD45+ Hi and CD45+ low populations may be separated using a cell sorter based on size and granularity.

The cytokine combinations useful in carrying out various embodiments of the invention may be used at any suitable final working concentration to achieve the desired effect. For example, BMP-4 may be used at a concentration ranging from about 1 ng/ml to about 120 ng/ml; from about 5 ng/ml to about 100 ng/ml; from about 10 ng/ml to about 80 ng/ml; from about 25 ng/ml to about 75 ng/ml; from about 30 ng/ml to about 60 ng/ml. In some embodiments of the invention about 50 ng/ml of BMP-4 may be used. VEGF may be used at a concentration ranging from about 1 ng/ml to about 120 ng/ml; from about 5 ng/ml to about 100 ng/ml; from about 20 ng/ml to about 80 ng/ml; from about 25 ng/ml to about 75 ng/ml; from about 30 ng/ml to about 60 ng/ml. In some embodiments of the invention about 50 ng/ml of VEGF may be used. GM-CSF may be used at a concentration ranging from about 1 ng/ml to about 120 ng/ml; from about 5 ng/ml to about 100 ng/ml; from about 20 ng/ml to about 80 ng/ml; from about 25 ng/ml to about 75 ng/ml; from about 30 ng/ml to about 60 ng/ml. In some embodiments of the invention about 50 ng/ml of GM-CSF may be used. SCF may be used at a concentration ranging from about 1 ng/ml to about 350 ng/ml; from about 5 ng/ml to about 300 ng/ml; from about 10 ng/ml to about 250 ng/ml; from about 15 ng/ml to about 200 ng/ml; from about 20 ng/ml to about 150 ng/ml; from about 5 ng/ml to about 50 ng/ml. In some embodiments of the invention about 20 ng/ml of SCF may be used. FLT3L may be used at a concentration ranging from about 1 ng/ml to about 350 ng/ml; from about 5 ng/ml to about 300 ng/ml; from about 10 ng/ml to about 250 ng/ml; from about 15 ng/ml to about 200 ng/ml; from about 20 ng/ml to about 150 ng/ml. In some embodiments of the invention about 20 ng/ml of FLT3L may be used. IL-3 may be used at a concentration ranging from about 1 ng/ml to about 80 ng/ml; from about 5 ng/ml to about 75 ng/ml; from about 10 ng/ml to about 50 ng/ml; from about 20 ng/ml to about 40 ng/ml. In some embodiments of the invention about 25 ng/ml of IL-3 may be used. TPO may be used at concentration ranging from about 1 ng/ml to about 150 ng/ml; from about 5 ng/ml to about 100 ng/ml; from about 10 ng/ml to about 80 ng/ml; from about 20 ng/ml to about 60 ng/ml. In some embodiments of the invention about 20 ng/ml of TPO may be used. IL-4 may be used at a concentration ranging from about 1 ng/ml to about 120 ng/ml; from about 5 ng/ml to about 100 ng/ml; from about 20 ng/ml to about 80 ng/ml; from about 25 ng/ml to about 75 ng/ml; from about 30 ng/ml to about 60 ng/ml. In some embodiments of the invention about 50 ng/ml of IL-4 may be used. The concentration may be any value or subrange within the recited ranges, including endpoints.

In some embodiments of the invention a maturation cocktail comprising a plurality of cytokines may be used to mature imDC to mDC. Suitable final working concentrations of cytokine components of the maturation cocktail may include any concentration which effectively matures imDC to mDC. For example IFNy may be used at a concentration ranging from about 1 ng/ml to about 150 ng/ml; from about 5 ng/ml to about 100 ng/ml; from about 10 ng/ml to about 100 ng/ml; from about 15 ng/ml to about 80 ng/ml; from about 20 ng/ml to about 60 ng/ml. In some embodiments of the invention about 25 ng/ml of IFNy may be used. In other embodiments of the invention about 10 ng/ml of IFNy may be used. In other embodiments of the invention about 5 ng/ml of IFNy may be used. TNFa may be used at a concentration ranging from about 1 ng/ml to about 200 ng/ml; from about 10 ng/ml to about 150 ng/ml; from about 20 ng/ml to about 100 ng/ml; from about 30 ng/ml to about 80 ng/ml; from about 40 ng/ml to about 75 ng/ml. In some embodiments of the invention about 10 ng/ml of TNFa may be used. IL-1β may be used at concentration ranging from about 1 ng/ml to about 200 ng/ml, from about 5 ng/ml to about 150 ng/ml; from about 8 ng/ml to about 75 ng/ml; from about 10 ng/ml to about 50 ng/m. In some embodiments of the invention about 10 ng/ml of IL-1β may be used. The concentration may be any value or subrange within the recited ranges, including endpoints.

PGE2 may be used at a concentration ranging from about 0.1 ug/ml to about 150 ug/ml; from about 0.5 ug/ml to about 100 ug/ml; from about 0.8 ug/ml to about 75 ug/ml; from about 1 ug/ml to about 50 ug/ml. In some embodiments of the invention about 1 ug/ml of PGE2 may be used. Poly I:C may be used a concentration ranging from about 1 ug/ml to about 50 ug/ml, from about 5 ug/ml to about 40 ug/ml from about 10 ug/ml to about 30 ug/ml, form about 15 ug/ml to about 25 ug/ml. In some embodiments of the invention about 20 ug/ml of Poly I:C may be used. The concentration may be any value or subrange within the recited ranges, including endpoints.

In certain embodiments the invention provides for the differentiation of hPS cells in hematopoietic lineage cells wherein at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of cells express one or more markers or factors that are expressed by cells of hematopoietic lineage. The concentration may be any value or subrange within the recited ranges, including endpoints.

Antigen-Presenting Cells

Antigen presenting cells of this invention are often referred to in this disclosure as “dendritic cells”. However, this is not meant to imply any morphological, phenotypic, or functional feature beyond what is explicitly required. The term is used to refer to cells that are phagocytic or can present antigen to T lymphocytes, falling within the general class of monocytes, macrophages, dendritic cells and the like, such as may be found circulating in the blood or lymph, or fixed in tissue sites. Phagocytic properties of a cell can be determined according to their ability to take up labeled antigen or small particulates. The ability of a cell to present antigen can be determined in a mixed lymphocyte reaction as described. Certain types of dendritic cells and antigen-presenting cells in the body are first identified in tissue sites such as the skin or the liver; but regardless of their origin, location, and developmental pathway, they are considered in the art to fall within the general category of hematopoietic cells. By analogy, the term dendritic cells used in this disclosure also fall in the broad category of hematopoietic cells, whether produced through the hematopoietic or direct paradigm framed earlier, or through a related or combined pathway.

The putative role of hPS derived cells as antigen-presenting cells is provided in this disclosure as an explanation to facilitate the understanding of the reader. However, the theories expostulated here are not intended to limit the invention beyond what is explicitly required. The hPS derived cells of this invention may be used therapeutically regardless of their mode of action, as long as they achieve a desirable clinical benefit in a substantial proportion of patients treated.

As such, antigen-presenting cells may include, but are not necessarily limited to, dendritic cells, macrophages, and B cells. In specific embodiments, the antigen-presenting cells are dendritic cells.

In some embodiments, the antigen expressing cells described herein express an antigen of an infectious agent. In some embodiments, the infectious agent is a virus, bacterium, parasite, protozoan, or a fungus.

In specific embodiments, the infection is caused by a virus. The virus may be a DNA virus, a RNA virus, or a retrovirus. Non-limiting example of viruses useful with the present invention include, but are not limited to Ebola, measles, SARS-CoV, SARS-CoV-2, Chikungunya, hepatitis, Marburg, yellow fever, MERS-CoV, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyoxivirus, Australian bat lyssavirus, Avian bornavirus, Avian metapneumovirus, Avian paramyoxviruses, penguin or Falkland Islandsvirus, BK polyomavirus, Bagaza virus, Banna virus, Bat hepevirus, Bat sapovirus, Bear Canon mammarenavirus, Beilong virus, Betacoronoavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwere virus, Caliciviridae virus. California encephalitis virus, Candiru virus, Canine distemper virus, Canaine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyoxiviurs SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human gential-associated circular DNA virus-1, Human herpesvirus 1-8, Human immunodeficiency virus 1/2, Huan mastadenovirus A-G, Human papillomavirus, Human parainfluenza virus 1-4, Human paraechovirus, Human picobirnavirus, Human smacovirus, Ikoma lyssavirus, Ilheus virus, Influenza A-C, Ippy mammarenavirus, Irkut virus, J-virus, JC polyomavirus, Japanses encephalitis virus, Junin mammarenavirus, KI polyomavirus, Kadipiro virus, Kamiti River virus, Kedougou virus, Khuj and virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\0.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Procine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS-Coronavirus, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In some embodiments, the infection is caused by a bacterium. Non-limiting examples of bacteria that can be useful in accordance with the disclosed methods include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Trichophyton rubrum, T. mentagrophytes, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

In some embodiments, the infection is caused by a parasite. Examples of parasites useful in accordance with disclosed methods include without limitation one or more of (or any combination of), an Onchocerca species and a Plasmodium species.

In some embodiments, the infection is caused by a protozoan. Examples of protozoa that can be useful in accordance with the disclosed methods and devices include without limitation any one or more of (or any combination of), Euglenozoa, Heterolobosea, Diplomonadida, Amoebozoa, Blastocystic, and Apicomplexa. Example Euglenoza include, but are not limited to, Trypanosoma cruzi (Chagas disease), T. brucei gambiense, T. brucei rhodesiense, Leishmania braziliensis, L. infantum, L. mexicana, L. major, L. tropica, and L. donovani. Example Heterolobosea include, but are not limited to, Naegleria fowleri. Example Diplomonadid include, but are not limited to, Giardia intestinalis (G. lamblia, G. duodenalis). Example Amoebozoa include, but are not limited to, Acanthamoeba castellanii, Balamuthia madrillaris, Entamoeba histolytica. Example Blastocystis include, but are not limited to, Blastocystic hominis. Example Apicomplexa include, but are not limited to, Babesia microti, Cryptosporidium parvum, Cyclospora cayetanensis, Plasmodium falciparum, P. vivax, P. ovale, P. malariae, and Toxoplasma gondii. Babesia microti, Cryptosporidium parvum, Cyclospora cayetanensis, Plasmodium falciparum, P. vivax, P. ovale, P. malariae, and Toxoplasma gondii.

In some embodiments, the infection is caused by a fungus. Examples of fungi that can be useful in accordance with the disclosed methods include without limitation any one or more of (or any combination of), Aspergillus, Blastomyces, Candidiasis, Coccidiodomycosis, Cryptococcus neoformans, Cryptococcus gatti, sp. Histoplasma sp. (such as Histoplasma capsulatum), Pneumocystis sp. (such as Pneumocystis jirovecii), Stachybotrys (such as Stachybotrys chartarum), Mucroymcosis, Sporothrix, fungal eye infections ringworm, Exserohilum, Cladosporium.

In certain example embodiments, the fungus is a yeast. Examples of yeast that can be detected in accordance with disclosed methods include without limitation one or more of (or any combination of), Aspergillus species (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), a Geotrichum species, a Saccharomyces species, a Hansenula species, a Candida species (such as Candida albicans), a Kluyveromyces species, a Debaryomyces species, a Pichia species, or combination thereof. In certain example embodiments, the fungi is a mold. Example molds include, but are not limited to, a Penicillium species, a Cladosporium species, a Byssochlamys species, or a combination thereof.

In specific embodiments, the antigen-expressing cells express an antigen of a Coronaviridae virus. In specific embodiments, the Coronaviridae virus is SARS-CoV-2. In specific embodiments, the Coronaviridae antigen comprises the Coronaviridae spike (S) protein. In specific embodiments, the Coronaviridae antigen comprises the full-length, 1,273 amino acid spike protein. In specific embodiments, the Coronaviridae antigen comprises the spike protein receptor-binding domain (RBD). In specific embodiments, the Coronaviridae antigen comprises a 209 amino acid sequence of the RBD. In specific embodiments, the Coronaviridae antigen comprises the spike protein S1 domain, S2 domain, or both. In specific embodiments, the Coronaviridae antigen comprises the nucleocapsid protein. In specific embodiments, the Coronaviridae antigen comprises the membrane protein.

Genetic Alterations

As used herein, “genetically altered”, “transfected”, or “genetically transformed” refer to a process where a polynucleotide has been transferred into a cell by any suitable means of artificial manipulation, or where the cell is a progeny of the originally altered cell and has inherited the polynucleotide. The polynucleotide will often comprise a transcribable sequence encoding a protein of interest, which enables the cell to express the protein at an elevated level or may comprise a sequence encoding a molecule such as siRNA or antisense RNA that affects the expression of a protein (either expressed by the unmodified cell or as the result of the introduction of another polynucleotide sequence) without itself encoding a protein. The genetic alteration is said to be “inheritable” if progeny of the altered cell have the same alteration.

The cells of this invention can also be genetically altered in order to enhance their ability to be involved in modulating an immune response, or to deliver a therapeutic gene to a site of administration. A vector is designed using the known encoding sequence for the desired gene, operatively linked to a promoter that is either pan-specific or specifically active in the differentiated cell type. Alternatively the promoter may be an inducible promoter that permits for the timed expression of the genetic alteration. For example the cells may be genetically engineered to express a cytokine that modulates an immune response either by enhancing the response or dampening the response.

In some embodiments of the invention, the cells are permanently transduced with a gene that enables the cells to express the gene product in progeny that bear characteristics of dendritic cells. The cells can be transduced while they are still undifferentiated hPS cells, or at an intermediate stage (such as a hematopoietic or dendritic cell precursor). Methods for genetically altering hPS cells in the presence or absence of feeder cells using lipofectamine are described in US 2002/0168766 A1 (Geron Corp.). Other transfections methods, including electroporation, may be used. Lentiviral and retroviral vectors are also suitable. Alternatively, the expression cassette can be placed into a known location in the genome of the cell by homologous recombination (US 2003/0068818 A1).

Genetic modifications that can promote the immunogenic effect include expression of cytokines such as IL-12 or IL-15 that contribute to cytotoxic T cell activation or memory, or chemokine equivalents such as secondary lymphoid tissue chemokine (SLC), IFNγ (which induces monokine), or lymphotactin (Lptn). Costimulatory molecules like B7 may enhance T cell activation. Inhibition of invariant chain expression (by knockout, antisense, or RNAi technology) may enhance the CD4+ T cell component of the response.

In some embodiments, the hPS cells are genetically altered before they are differentiated.

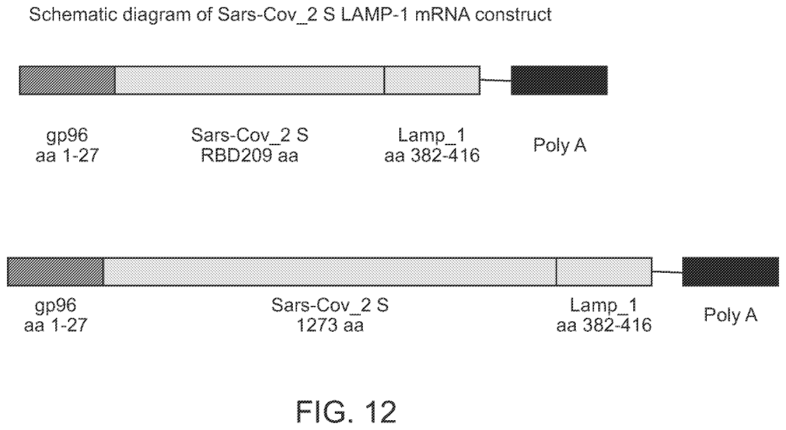

In some embodiment, the hPS cells are genetically altered after they are differentiated. In some embodiments, the hPS are genetically altered by transfecting them with a polynucleotide encoding a Coronaviridae virus antigen and the cytoplasmic tail of lysosomal associated membrane protein 1 (LAMP-1). In specific embodiments, the polynucleotide further comprises a poly(A) tail and a gene which encodes heat shock protein 96 (HSP96), as described in the examples and in FIG. 12.

Formulations

When intended for clinical use, the dendritic cell preparations described in this disclosure are formulated for administration to a human subject. This means that the cells are prepared in compliance with local regulatory requirements, are sufficiently free of contaminants and pathogens for human administration, and are suspended in isotonic saline or other suitable pharmaceutical excipient.

mDC according to this invention may be administered to a human subject to stimulate an immune response in the subject. Prior to administration, the imDC may be contacted with an antigen of interest and then matured into mDC. The antigen may be internalized and processed such that it is presented on the cell surface in the context of MHC I and/or MHC II and thus may stimulate a specific immune response to the antigen. In some embodiments the specific immune response may have a therapeutic effect. In other embodiments the immune response may provide a prophylactic effect. In still other embodiments the specific immune response may provide a source of antigen specific cells such as cytotoxic T cells, or B lymphocytes or antibodies which specifically recognize the antigen. Administration of the cells according to the invention may be by intravenous, intradermal or intramuscular injection. In other embodiments the cells may be administered subcutaneously. The cells may be formulated with an appropriate buffer, such as PBS and/or an appropriate excipient. The cells may be formulated with a suitable adjuvant. Examples of suitable pharmaceutical carriers are described in Remington's Pharmaceutical Sciences by E. W. Martin 20th Edition. Baltimore, Md.: Lippincott Williams & Wilkins, 2000.

For general principles in medicinal formulation and use of cellular vaccine compositions, the reader is referred to Handbook of Cancer Vaccines by M. A. Morse et al., Humana Press, 2004; Cancer Vaccines and Immunotherapy by P. L. Stern et at, eds., Cambridge Univ. Press, 2000; and the most recent edition of Good Manufacturing Practices for Pharmaceuticals by S. H. Willig, Marcel Dekker. The testing and use of dendritic cell vaccines is reviewed in the reference texts Dendritic Cell Protocols (Methods in Molecular Medicine, 64) by S. P. Robinson et al., Humana Press, 2001; and Dendritic Cells in Clinics by M. Onji, Springer-Verlag, 2004.

Any of the dendritic cell preparations of this invention (precursors or mature, immunogenic or toleragenic, and if immunogenic, before or after loading with antigen) can be stored after preparation to be used later for therapeutic administration or further processing. Methods of cryoconserving dendritic cells both before and after loading are described in PCT publication WO 02/16560 (B. Schuler-Thurner et al.).

Occasional reference to a pharmaceutical composition in this disclosure as a “vaccine” implies no particular mode of action or administration. The term means only that it has been formulated for administration to a human subject as already described. A vaccine may be designed as an immunogenic composition for generating a CTL response against a target antigen—but this need not be demonstrated as long as the composition is therapeutically effective according to any suitable clinical criterion in a reasonable proportion of treated cancer patients.

Various cell preparations of this invention can be maintained or supplied in combination with each other or with materials useful in their manufacture or use. Commercial embodiments include any system or combination of cells or reagents that exist at any time during manufacture, distribution, testing, or clinical use of the hPS derived dendritic cells, as described in this disclosure. Cell populations that may be useful together are undifferentiated hPS cells, hPS-derived dendritic cell precursors, mature dendritic cells, toleragenic dendritic cells, or other differentiated cell types, in any combination, sometimes derived from the same hPS cell line.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration can include powders or granules, suspensions or solutions in water or non-aqueous media, capsules, sachets, or tablets. Thickeners, flavorings, diluents, emulsifiers, dispersing aids or binders can be desirable.

In some embodiments, various cell preparations described herein can be maintained or supplied in combination with each other or with materials useful in their manufacture or use. Commercial embodiments include any system or combination of cells or reagents that exist at any time during manufacture, distribution, testing, or clinical use of the hPS derived dendritic cells, as described in this disclosure. Cell populations that may be useful together are undifferentiated hPS cells, hPS-derived dendritic cell precursors, mature dendritic cells, toleragenic dendritic cells, or other differentiated cell types, in any combination, sometimes derived from the same hPS cell line.

Pharmaceutical compositions of this invention may optionally be packaged in a suitable container with written instructions for a desired purpose, such as the reconstitution of hematopoietic-lineage cell function to improve a disease condition or to stimulate an immune response.

Administration

The compounds and pharmaceutical compositions described herein can be administered to the subject in a number of ways depending on whether local or systemic treatment is desired, and on the area to be treated. Thus, for example, a compound or pharmaceutical composition described herein can be administered as an ophthalmic solution and/or ointment to the surface of the eye. Moreover, a compound or pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

### III. Compositions

In embodiments, the methods provided herein include a composition for treating an infectious disease. The composition may comprise a formulation of differentiated hPS cells as described elsewhere herein. The hPS may be differentiated into a population of mature antigen-presenting cells, wherein said antigen expressing cells express an antigen of an infectious agent, as described elsewhere herein. The differentiated hPS cells may also be genetically altered before or after they are differentiated so that they express a protein comprising one or more immunogenic epitopes of the infectious agent. The composition may then be administered to a human for preventing an infectious disease.

As described elsewhere herein in detail, the cellular compositions are made from stem cells, particularly pluripotent stem cells of human origin. The culture is differentiated into cells having characteristics of antigen-presenting cells, loaded with a specific target antigen on an infectious particle, and formulated for administration to a human subject. The differentiation process involves culturing the cells in an environment of cytokines and other factors that generate a hematopoietic or early dendritic cell progenitor, and then maturing the cells to the phenotype intended for administration. Effective factor combinations and markers to effect and monitor the differentiation procedure are provided elsewhere in the disclosure.

In some embodiments, the antigen-presenting cells are selected from the group consisting of dendritic cells, macrophages, B cells, and a combination thereof.

In specific embodiments, the antigen-presenting cells are dendritic cells.

In some embodiments, the infectious disease is a viral infection caused by a virus.

In some embodiments, the antigen-presenting cells express an antigen of said virus.

In some embodiments, the virus is a Coronaviridae virus. In specific embodiments, the Coronaviridae virus is SARS-CoV-2.

In some embodiments, the composition is administered to a human for treating an infectious disease.

In some embodiments, the composition is administered to a human for preventing an infectious disease.

In some embodiments, the composition is a vaccine.

In other embodiments, the disclosure also provides a composition comprising dendritic cells expressing a Coronaviridae antigen. In some embodiments, the Coronaviridae antigen comprises the Coronaviridae spike protein.

In some embodiments, the composition comprises the whole, 1,273 amino acid spike protein. In some embodiments, the composition comprises the spike protein receptor-binding domain (RBD). In some embodiments, the Coronaviridae antigen comprises a 209 amino acid sequence of the RBD.

In some embodiments, the Coronaviridae antigen comprises the spike protein S1 domain, S2 domain, or both.

In some embodiments, the composition comprises the Coronaviridae nucleocapsid protein.

In some embodiments, the composition comprises the Coronaviridae membrane protein.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Engineer DC Cells with a Relevant SARS-CoV-2 Antigen

The workhorse tool to modulate or prepare an immune system to fight aggressing pathogens is the vaccine. This approach consists of presenting antigens from pathogens to our immune system such that we prepare defense mechanisms ahead of a future infection to prevent it from taking hold in our body. Although there have been many improvements in vaccine research, the vaccine development timeline is often outpaced by the propagation of emerging diseases, adding substantial pressure to healthcare systems. This effectively limits their utilities in the early phase of disease propagation.

Adoptive immunotherapy is a novel type of therapeutic approach that has shown tremendous potential in fighting cancer. Adoptive immunotherapy generally consists of engineering the effector cells of our immune system (DCs and T Cells) to respond by detecting and destroying cancer cells. Similar to traditional vaccines, one limitation of cancer vaccines is that it usually takes time to select a relevant target antigen to fight a specific disease and engineer large cell banks to address broad segment populations. Applicants developed a unique approach to cancer vaccines, which could potentially address development timelines and readiness.

Competing approaches (aptly referred to as “personalized medicine”) rely mostly on engineering a patient's own immune cells such at T Cells as a first step, followed by expansion and reinfusion of the modified cells within the same patient. Applicants' strategy is built on its ability to produce large cell banks from pluripotent universal donor cells that are terminally differentiated into DCs, the main APC of the immune system. Such cell banks can then be modified with a relevant antigen shortly prior to injecting patients to elicit an immune response, a concept called a DC Vaccine. This fundamental difference could greatly accelerate development and improve accessibility for broad patient populations. Such a “plug-and-play” approach can be tailored initially to express SARS-CoV-2 relevant antigens to treat COVID-19, while large DC banks can be stored and modified quickly with future emerging diseases antigens.

TABLE 1

| | Timeline to | Addressable patient |
|---|---|---|
| Therapeutic approach | clinical trial | population |
| DC vaccine | Fast | +++ |
| TCR/CAR-T cell | Slow | + |
| Traditional vaccines | Slow | +++ |
| Novel vaccine platform | Medium | +++ |

The platform technology is already being tested in a Phase 1 clinical trial of patients with non-small cell lung cancer by Cancer Research UK. In this trial, DCs have been engineered to express the tumor-selective antigen telomerase.

Phase I: DC cells will be engineered with a relevant SARS-CoV-2 antigen such as the Spike protein for downstream testing in a small clinical study. Applicants already benefit from fully characterized human pluripotent cell banks, robust process development, quality assurance and regulatory teams, and validated clean rooms for cGMP production.

Phase II: In parallel to preclinical testing requirements necessary to initiate human trials, Applicants will work on scale up processes to establish methods and pathways to bring such therapy to large scale population. This will demonstrate an ability to address future large scale needs for emerging threats.

From a therapeutic perspective, DC vaccines offer a unique opportunity for a “One-Two-Punch” for COVID-19 and other emerging diseases. As the main APC effector in humans, DCs can act not only on activation of killer T cells and macrophages to eliminate ongoing infections, but also could play a role in the preservation of an immune memory for future infections. At this stage, it is difficult to predict if mounting an immune reaction could exacerbate symptoms in a diseased population. To mitigate this, trials will be designed in both diseased and naïve patient population.

A key technical risk resides on the effectiveness of any given antigen such as the Spike protein of SARS-CoV-2 to activate a relevant immune response. As such, use of a combination of antigens might lead to an improved therapeutic approach. The platform described herein allows for the simultaneous introduction of various antigens within the same batch of DC for multiple target presentation.

### Example 2: Dendritic Cell Vaccines for Cancer Treatment and COVID-19 Prevention

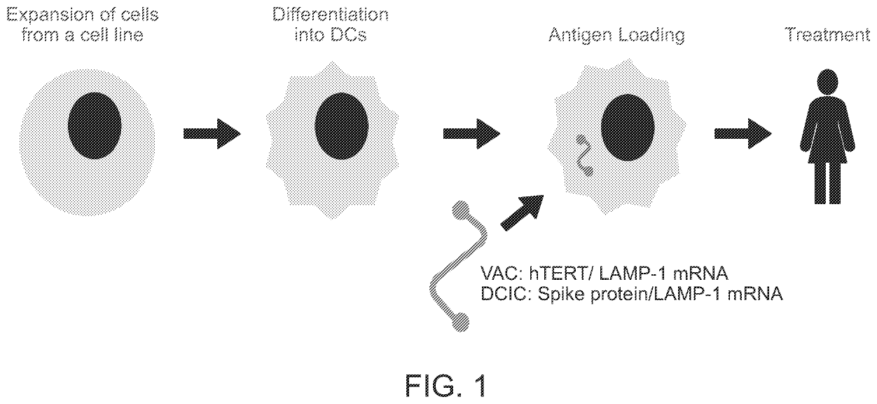

The Dendritic Cell Infectious Disease Program builds on the previously described VAC Platform using mature dendritic cells to increase a patient's immune response. The dendritic cell vaccine is an allogenic (“off the shelf”) vaccine. Cells are manufactured from a pluripotent cell line and not derived from the patient. This provides advantages in terms of time and cost. Mature dendritic cells are manufactured and loaded with relevant antigen to stimulate CD8+ (cytotoxic) and CD4+ (helper) T cell responses. “Targeted education” of T cells increases immune response and pathogen destruction (FIG. 1).

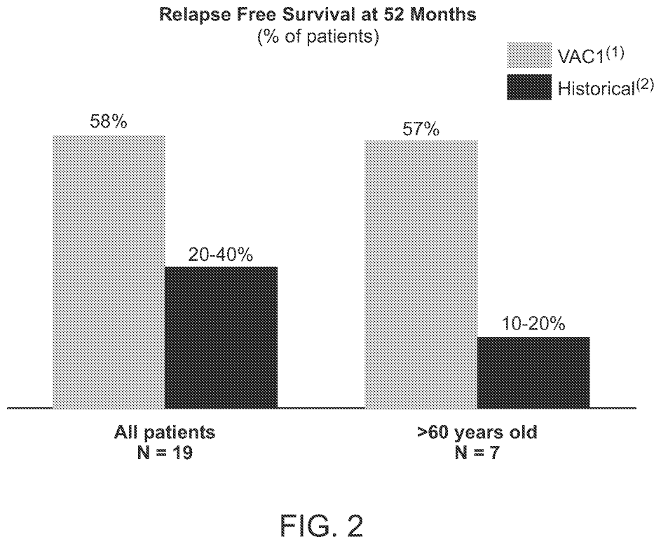

FIG. 2 highlights results from the Phase II, multicenter, open-label trial. 19 patients with acute myeloid leukemia (AML) were treated and they ended up in complete remission. These were intermediate and high risk patients, evaluated by cytogenetics. Seven of these treated patients were in a very high risk group, over the age of 60. The treatment showed robust safety in all patients treated, with a clear anti-telomerase immune response.

The VAC1 program involved autologous dendritic cell therapy for AML. hTERT-specific T cell responses were induced in 58% of patients. 58% remained in cancer remission at the median duration of follow-up of 52 months from first vaccination. The response was reproducible: 3 of 4 patients exhibited increased hTERT-specific T cells by pentamer assay that coincided with their dendritic cell vaccination schedule. The percentage of hTERT-specific T cells detected by pentamer staining ranged from 0.75% to 3.78% in patients treated to date, similar to other approved cancer vaccines. The frequency of antigen-specific T cells required for immunity against infectious diseases can be as low as 0.1% (typically 0.2-0.6%) of the total T cell population. Based on these findings, the magnitude of the hTERT-specific T cell response generated by VAC2 is viewed as being of potential biological relevance and warrants further investigation. There is also evidence of durability: for the two VAC2 patients with pre- and post-vaccination pentamer assay data, the apparent sustained increase in hTERT-specific T cells provides evidence of a durable immune response.

Dendritic cell vaccines allow for rapid deployment to fight the COVID-19 pandemic and prepare for future emerging threats.

Preparedness and Surveillance. A dendritic cell bank and arsenal can be built now to Produce cGMP grade fully tested pluripotent cell banks. In house development and manufacturing operations allow for large scale differentiated dendritic cells (drug substance). Ongoing and emerging threats can then be monitored for relevant antigens.

Fast Response. Upon identification of a new emerging threat, dendritic cells can be armed with the relevant antigen. The cGMP antigen can be generated—any type of antigen is possible, including peptides, protein, oligonucleotides, or mRNA. The antigen can be electroporated into the dendritic cells, and stocks can be cryopreserved and deployed to repositories. Dendritic cell vaccines are formulated and cryopreserved for simple thawing and injection.

Deployment. The vaccines can be distributed and administered. The ready to use vaccines can be sent to repositories and treatment centers, stored frozen until use, then thawed, loaded into syringes and administered by intradermal injection.

Potential advantages of the dendritic cell vaccine approach include the existence of a single master cell bank for scalability and consistency, the fact that the cells are quickly tailorable and adaptable to any antigen, the therapy is available off the shelf, there are lower anticipated costs compared to CAR-T therapy, it can be used in combination with other modalities, it can be used to potentially treat an ongoing infection, and it provides immune memory to future infection.

Furthermore, dendritic cell therapy is a unique opportunity to complement ongoing efforts to fight COVID-19. The availability of cGMP cell banks permits rapid development of clinical grade material for human testing. The availability of relevant (or suspected) antigen targets can be uploaded quickly into naïve dendritic cells. The safety profile in humans has been established in cancer immunotherapy clinical trials. The pathway from clinical material availability to human trials is fast—it only requires an in vitro bioassay because efficacy testing in animals is not possible. This system could provide tools to both fight active COVID-19 infection and provide multi-year memory immunity. It is broadly applicable to any infectious disease. This product could play a critical role in certain settings, genetic or professional, due to its differentiated approach to both treating disease and providing lasting immunity.

### Example 3: Expression of mRNA in Dendritic Cells

In this study, dendritic cells were derived from monocytes extracted from peripheral blood mononuclear cells, which were collected and cultured in a manner that enriches for antigen-presenting cells. Purified dendritic cells were then electroporated with mRNA coding for GFP or SARS-CoV-2 Spike protein. Expression of the spike protein was assessed 16 hours post-electroporation by intracellular staining of CD83, CD86, HLA-DR, followed by flow cytometry. Percent expression of the spike and GFP proteins is shown in Table 2. Monocytes were differentiated to mature dendritic cells as expected showing high expression of CD83, CD86, and HLA-DR (>90%). CD83 is a marker for dendritic cell maturation, while CD86 and HLA-DR are costimulatory molecules involved in T cell activation.

TABLE 2
Culturing and electroporation conditions

| Condition | | | | mRNA volume added | | Electroporation | |
|---|---|---|---|---|---|---|---|
| # | Group | Cell type | mRNA | (0.5 μg/400K cells) | | +/− | #Cells |
| 1 | Control (1) | Transfected | Lentivirus- | NA | | − | 1 × 10⁶ |
| | | HEK 293T cells | SARS-CoV-2 spike | | | | |
| 2 | Control (2) | Monocyte- | NA | NA | | − | 0.5 × 10⁶ |
| | | derived mDCs | | | | | |
| 3 | Control (3) | | GFP 2 μg/μl | 0.125 | μl | + | 0.4 × 10⁶ |
| 4 | Control (4) | | SARS-CoV-2 spike | 2 | μl | − | 0.8 × 10⁶ |
| | | | 2.4 μg/μl | | | | |
| | | | (Invitrogen kit) | | | | |
| 5 | A | | SARS-CoV-2 spike | 2 | μl | + | 0.8 × 10⁶ |
| | | | 2.4 μg/μl | | | | |
| | | | (Invitrogen kit) | | | | |
| 6 | B | | SARS-CoV-2 spike | 2.4 | μl | + | 0.8 × 10⁶ |
| | | | 2.4 μg/μl | | | | |
| | | | (NEB kit) | | | | |

293T cells were transfected with SARS-CoV-2 spike protein. H1 cells were used as a negative control. Groups in Table 2 were as follows. Control (2) consisted of fresh mDCs that were not electroporated, control (4) consisted of fresh mDCs with spike mRNA that were not electroporated, (A) consisted of fresh mDCs with spike mRNA (Invitrogen) that were electroporated, and (B) consisted of fresh mDCs with spike mRNA (NEB) that were electroporated.

TABLE 3
Cells used

| Sample | Related test # or Vial identifier |
|---|---|
| #1 | Monocytes isolated from buffy coat of |
| | healthy donors (V-MO-20-001)- test |
| #2 | Human embryonic stem cells H1 - negative |
| | control |
| #3 | HEK 293T transfected with |
| | Lentivirus vector SARS CoV-2 Spike mRNA- |
| | positive control |

TABLE 4
Equipment

Equipment
BS cabinet
Incubator for 37° C. ± 1° C. 5% CO2
NC-200 cell counter, Chemometec
4D X Nucleofector, Lonza
Centrifuge
Pipette Aid
Pipettor 1-10 μl
Pipettor 5-50 μl
Pipettor 20-200 μl
Pipettor 100-1,000 μl
CytoFlex Flow cytometer, Beckman Coulter

TABLE 5
Materials

| Material | Manufacturer | Cat # |
|---|---|---|
| P3 Primary Cell 4D X kit S | Lonza | V4XP-3032 |
| Heat Inactivated Fetal Bovine | BI | 04-127-1A |
| Serum (FBS) | | |
| CellGenix DC complete medium | Cellgenix | 20801-0500 |
| P/S | BI | 03-031-1B |
| L-glutamine | BI | A03-020-1 |
| DMEM F12 medium | Gibco | 10565-018 |
| Sterile tissue culture 6-well plate | Corning | |
| 24-well ULA plate | Corning | |
| EGFP (capG) mRNA 2.0 μg/μl, | NA | NA |
| 240 μg | | |
| mRNA Covid-19 full-length spike | NA | NA |
| protein 2.4 μg/ul (Invitrogen kit) | | |
| mRNA Covid-19 full length spike | NA | NA |
| protein 2.0 μg/ul (NEB kit) | | |
| Via1 cassette | Chemometech | 941-0011 |
| GM-CSF | Cellgenix | 001012-1000 |
| IL-4 | R&D System | 204-GMP-050 |
| IFNγ | R&D System | 285-GMP-100 |
| IL-1β | R&D System | 201-GMP-100 |
| TNF-α | R&D System | 210-GMP-100 |
| PGE2 | Sigma Aldrich | P6532 |
| DPBS (−) | BI | A02-023-1 |
| DPBS (+) | HyClone | SH30264.01 |
| EDTA 0.5M pH = 8.0 | BI | 01-862-1B |
| Thaw Buffer (PBS(−)/10% FBS HI) | Prepared by | NA |
| | user | |
| FACS buffer (PBS(−)/2% FBS HI/ | Prepared by | NA |
| 0.05% NaN₃) | user | |
| 70 μm cell strainer | Corning | 431751 |
| Foxp3 Fix/Perm kit | Invitrogen | 00-55-23-00 |
| Cytoflex QC beads | Rhenium | B53230 |
| FVS 450 | BD | 565388 |
| SARS-CoV-2 (2019-nCoV) Spike | SinoBiological | R007-40150 |
| S1 Antibody, Rabbit MAb | | |
| Goat anti-Rabbit Alexa fluor 647 | Jackson | 144-606-111 |
| | ImmunoResearch | |
| Anti-human CD83 APC | BD | 551073 |
| Anti-human CD86 PE | Biolegend | 305406 |
| Anti-human HLA-DR FITC | Milenyi Biotec | 130-111-788 |
| SpeI restriction enzyme | NEB | R3133S |
| mMESSAGE mMACHINE ™ | Invitrogen | AM1344 |
| T7 Transcription Kit | | |
| HiScribe T7 mRNA kit | NEB | E2060 |
| Megaclear Kit | Invitrogen | AM1908 |

Methods

Isolation of monocytes from buffy coat donated by healthy donors. Monocytes were isolated from buffy coat according to V-MO-20-001. Briefly:
Buffy coat was provided by the Israeli national blood bank.
Monocytes were isolated from buffy coat using RosetteSep™ Human Monocyte Enrichment Cocktail (Stem Cell Technologies, #Cat: 15068) and centrifugation over a buoyant density medium Lymphoprep™ (Stem Cell Technologies, #Cat: 07851).
Monocytes were cryopreserved in CS-10 and frozen in vapor phase of nitrogen for further use.

Generation of monocyte-derived mature dendritic cells. Monocyte-derived dendritic cells were generated according to V-MD-20-003. Briefly:
Monocytes were cultured on ultra low attachment T75 flasks (Corning) in CellGenix DC supplemented with GM-CSF and IL-4. Medium was replaced on day 1 and day 4.
On day 6, monocytes were differentiated into immature dendritic cells (iDC) and were stimulated with CellGenix DC medium supplemented with GM-CSF, TNF-α, IFN-γ, IL-1β, and PGE2 for additional 24 hours for generation of mature dendritic cells.

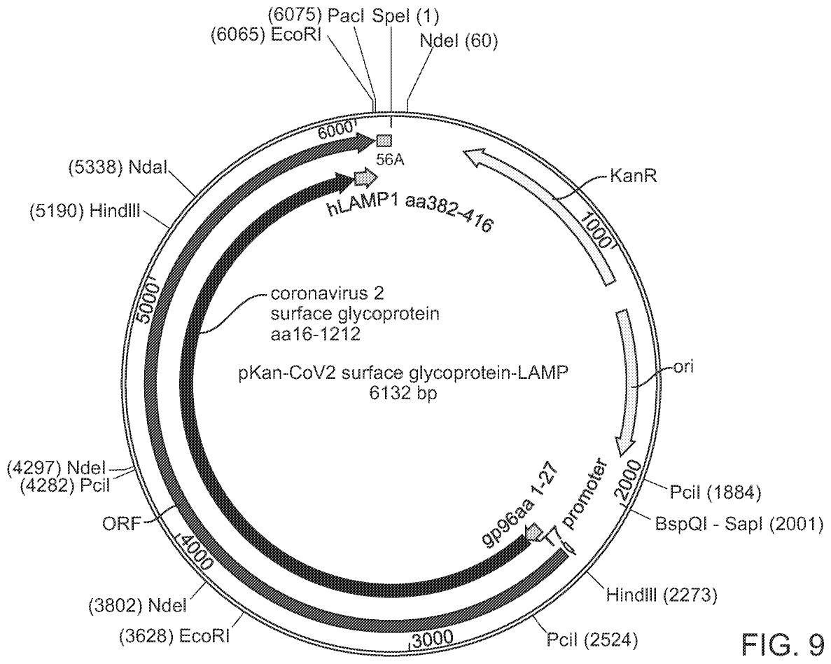

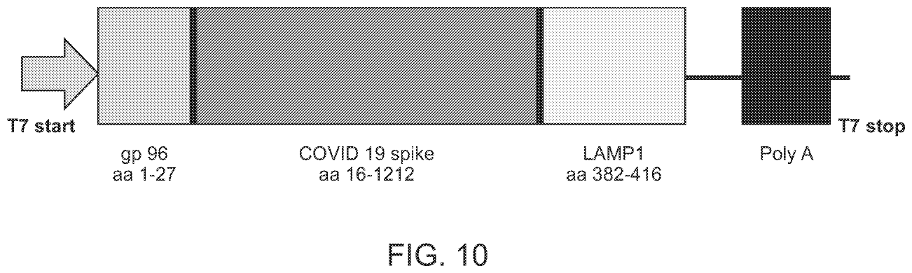

Plasmid DNA SARS-CoV-2 construction and mRNA synthesis. Plasmid DNA containing SARS-CoV-2 surface glycoprotein was constructed by cloning SARS-CoV-2 full length spike protein sequence (aa16-1212) into the pKAN-hTERT-LAMP1 as a vector backbone where the hTERT sequence was removed and swapped with the SARS-CoV-2 spike protein sequence (FIGS. 9 and 10).

To construct the mRNA, DNA fragments that encode the NH2-terminal signal peptide sequence (amino acids 1-27) of the heat shock protein 96 (gp96) and the endosomal/lysosomal targeting signal (amino acids 382-416) of LAMP-1 were ligated into restriction sites present in the SARS-CoV-2 spike protein. The SARS-CoV-2/COVID 19/LAMP-1 mRNA was under the control of the bacteriophage T7 promoter.

mRNA was synthesized from the plasmid DNA using Invitrogen mRNA synthesis kit mMES SAGE mMACHINE™ T7 Transcription Kit, Cat #AM1344 as well as HiScribe T7 ARCA mRNA kit (with tailing) Cat #E2060. Briefly:

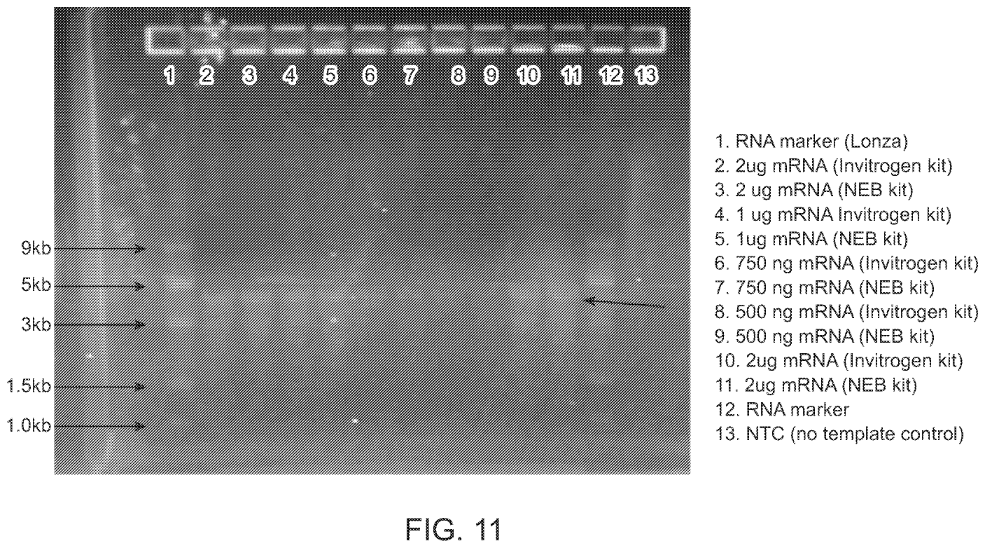

Plasmid DNA was linearized using restriction enzyme SpeI. Linearized plasmid DNA was purified using 1/20^{th} volume of 0.5M EDTA, 1/10^{th} volume of 3M Na acetate and 2 volume of ethanol. The plasmid DNA was resuspended in Tris EDTA buffer pH7.5-8.0. Reactions for mRNA synthesis were assembled at room temperature. For a 20 ul volume, 2×NTP/CAP was added, as well as 10× reaction buffer, linearized template DNA and enzyme mix. The reaction was mixed thoroughly by gently flicking the tube and by short centrifugation. The reaction mix was incubated at 37° C. for 1 hr. After 1 hr, TURBO DNase was added to the reaction mix to degrade the residual DNA at 37° C. for 15 min. Synthesized mRNA was purified using MEGAclear™ transcription clean up kit. Briefly:
Synthesized mRNA was brought to 100 ul by adding Elution buffer, then added 350 ul binding solution and 250 ul 100% ethanol. Mixed gently by pipetting.
The reaction mix was passed through the filter cartridge and washed twice with wash solution. The mRNA was eluted from the filter cartridge using 50 ul elution buffer. Eluted mRNA was quantified using Nanodrop.
Eluted mRNA had a concentration of 2062.6 ng/ul and the purity ratio A260/280 was 2.35 and A260/230 was 2.63.
The size of the mRNA was confirmed by running the mRNA on the denaturing RNA agarose gel from LONZA.
As expected, the purified SARS-CoV-2 (COVID 19) spike protein/LAMP-1 mRNA size was 4 kb, which includes a signal sequence 81 bp+full length covid spike protein 3819 bp+Lamp1 102 bp (FIG. 11).

Dendritic cell preparation for electroporation. Dendritic cell preparation was done according to the method procedure protocol. Briefly:
Cells were counted using NC-200 and “control (2)” and “control (4)” conditions, and were seeded according to the concentration mentioned in Table 2 in 24-well ULA plate, containing the final 0.8 ml CellGenix DC complete medium with mDC maturation factors cocktail.
Conditions “control (3)”, “A” and “B” (Table 2) were divided into three marked Eppendorf tubes, centrifuged, and cell pellets resuspended in 40 μl P3 electroporation in the final cell concentration of 10×10⁶/ml.
An aliquot of EGFP (capG) mRNA 2.0 μg/μl (stored at −80) was thawed on ice and diluted 1:8 (2 μl EGFP stock+14 μl P3) in P3 solution to a final concentration of 0.25 μg/μl.
An aliquot of mRNA SARS-CoV-2 full-length spike protein 2.4 μg/ul (Invitrogen kit) and mRNA SARS-CoV-2 full-length spike protein 2.0 μg/ul (NEB kit) were thawed on ice and diluted 1:9.6 (2 μl Invitrogen stock+17.2 μl P3) and 1:8 (2 μl NEB stock+14 μl P3) respectively in P3 solution to a final concentration of 0.25 μg/μ1.
Volumes of diluted mRNA were added to each cell suspension according to Table 2.
Cells were mixed and 20 μl of cell suspension was transferred into the electroporation cuvettes.
Cells were electroporated using the Nucleofector 4D X program SB-150.
Cells from each condition were seeded in a well of 24-well ULA plate containing the final 0.8 ml CellGenix DC complete medium with mDC factors.
Cells were incubated at 37° C. for 24 hours followed by flow cytometry assessment of DC maturation markers and SARS-CoV-2 Spike expression.

Preparation for flow cytometry. Dendritic cell preparation for flow cytometry was done according to method procedure protocol. Briefly:
Cells were stained with FVS viability staining.
For surface staining (HLA-DR, CD83 and CD86), cells were stained with primary conjugated antibody in FACS buffer followed by flow cytometry analysis.
For intra-cellular staining (Spike protein)-cells were fixed and permeabilized with Foxp3 Fix/Perm kit followed by staining with primary antibody against Spike protein (0.4 μg/test) and staining with secondary antibody (Goat anti-Rabbit) at a 1:200 dilution.
30,000 live single gated cells were acquired per tube using Cytoflex Flow Cytometer. When cell number was lower, all live cells in the sample were acquired.

Results

TABLE 6
Cell counts and viability before and post-electroporation

| | | | | | | | | % |
|---|---|---|---|---|---|---|---|---|
| | | | | | | % | | Cells in |
| | | | | | % Cells in | Viability | Live cells | aggregates |
| Condition | | | % Viability | Live cells | aggregates | 24 hrs after | 24 hrs after | 24 hrs after |
| # | Group | Cell type | Before Ele. | Before Ele. ×10⁶ | Before Ele. | seeding. | seeding ×10⁶ | Ele. |
| 1 | Control (1) | Transfected | NA | | | 76% | 1.25 × 10⁶ | 1% |
| | | 293T | | | | (post thaw) | | |
| 2 | Control (2) | Thawed monocyte- | 95.4% | 1.84 × 10⁶ | 0.5% | 95% | 0.18 × 10⁶ | 0% |
| | | derived mDCs | | | | | | |
| 3 | Control (3) | | | | | 81% | 0.04 × 10⁶ | 0% |
| 4 | Control (4) | | | | | 90% | 0.33 × 10⁶ | 2% |
| 5 | A | | | | | 94% | 0.27 × 10⁶ | 0% |
| 6 | B | | | | | 93% | 0.14 × 10⁶ | 2% |

TABLE 7
Flow cytometry results

| | | | | | % Live | | | | | |
|---|---|---|---|---|---|---|---|---|---|---|
| Condition | | | | EP | cells | | | | | |
| # | Group | Cell type | mRNA | +/− | (FVS450) | % GFP | % CD83 | % CD86 | % HLA-DR | % Spike |
| 1 | Control (1) | Transfected | Lentivirus-SARS | − | 71.9 | 2.66 | NA | 0 | NA | 21.2 |
| | | HEK 293T (PC) | Cov-2 Spike | | | | | | | |
| 2 | Control (2) | Monocyte- | NA | − | 89 | NA | NA | 99.6 | NA | 1.72 |
| 3 | Control (3) | derived mDCs | GFP | + | 93.1 | 60.7* | NA | NA | NA | NA |
| 4 | Control (4) | | SARS Cov-2 Spike | − | 83.6 | NA | 98.2 | 99.2 | NA | 1.06 |
| | | | (Invitrogen kit) | | | | | | | |
| 5 | A | | SARS Cov-2 Spike | + | 91.3 | NA | NA | 99.8 | 99.6 | 5.81 |
| | | | (Invitrogen kit) | | | | | | | |
| 6 | B | | SARS Cov-2 Spike | + | 88.9 | NA | NA | 99.7 | NA | 1.16 |
| | | | (NEB kit) | | | | | | | |
| NA | NA | hESC H1 (NC) | NA | NA | 84.2 | NA | 0.68 | 0.045 | 0 | 0.23 |

*low cell number

Buffy coat derived monocytes were differentiated to mature DCs showing high expression of CD83, CD86 and HLA-DR biomarkers (>95%). Monocyte-derived mature dendritic electroporated with eGFP mRNA were 60.7% positive for GFP expression 16 hours post electroporation. Positive control HEK 293T transiently transfected with lentivirus vector of SARS-CoV-2 Spike were 21.2% positive for spike protein. Negative control H1 hESC were 0.23% positive for spike protein. Monocyte-derived mature dendritic cells not electroporated with SARS-CoV-2 Spike mRNA or incubated with SARS-CoV-2 Spike mRNA without electroporation were 1.72% or 1.06% positive for spike, respectively (baseline). Monocyte-derived mature dendritic cells electroporated with SARS-CoV-2 Spike mRNA generated using the Invitrogen IVT kit were 5.81% positive for spike protein 16 hours post electroporation. Monocyte-derived mature dendritic cells electroporated with SARS-CoV-2 Spike mRNA generated using the NEB IVT kit were 1.16% positive for spike protein (same as the baseline) 16 hours post electroporation.

### Example 4: DNA Construct for In Vitro Transcription

A DNA construct for in vitro transcription is designed and optimized for antigen presentation. The construct is based on that previously used in the VAC1 and VAC2 clinical trials which utilized hTERT as the antigen. The mRNA encoded includes portions of the Spike sequence and other SARS-CoV-2 sequences yet to be determined and the cytoplasmic tail of lysosomal associated membrane protein 1 (LAMP-1), which facilitates human leukocyte antigen HLA II, as well as HLA I loading of target protein. Both elements are fused to the sequence encoding heat shock protein 96 (HSP96) to ensure that the protein translated from the mRNA construct is efficiently shuttled to the endoplasmic reticulum and subsequently expressed on the cell surface. Two candidate mRNA sequences are planned based on literature and other ongoing vaccine programs: Sars-COV-2S (RBD domain 209 aa sequence ID 209 aa and Sars-COV-2S (1,273 aa) (FIG. 12).

With several membrane proteins, antigen/LAMP chimeras were found to elicit a much greater immune response than wild-type antigen. This approach has proved useful in increasing cellular and humoral responses to several virus antigens, including human papillomavirus E7, dengue virus membrane protein, hepatitis C virus NS3 protein and cytomegalovirus pp65 (see, e.g., Bonini, et al., J Immunol. 166:5250-5257; 2001). The enhanced immune response can be attributed to co-localization of LAMP with MHC II and the more efficient processing and delivery of antigenic peptides. In addition, LAMP-targeting is reported to result in the presentation of an increased number of immunogenic epitopes, thus inducing a qualitatively broadened immune response compared to untargeted antigen. For example, Fernandes et al., (Eur J Immunol 30(8):2333-2343; 2000) demonstrated an increase in the number of presented peptides of a LAMP-trafficked OVA antigen encoded in a vaccinia vector. Of 12 peptides generated from exogenously supplied OVA, 9 were presented by an OVA/LAMP chimera, as compared to only 2 by the construct without LAMP.

### REFERENCES

1. Siddell, S.; Ziebuhr, J.; Snijder, E J. Coronaviruses, toroviruses, and arteriviruses. Vol. 1. London: Hodder Arnold; 2005. p. 823-856.
2. Weiss S R, Navas-Martin S. Coronavirus pathogenesis and the emerging pathogen severe acute respiratory syndrome coronavirus. Microbiology and molecular biology reviews: MMBR. 2005; 69:635-664. [PubMed: 16339739]
3. Heugel J, Martin E T, Kuypers J, Englund J A. Coronavirus-associated pneumonia in previously healthy children. The Pediatric infectious disease journal. 2007; 26:753-755. [PubMed: 17848893]
4. Kuypers J, Martin E T, Heugel J, Wright N, Morrow R, Englund J A. Clinical disease in children associated with newly described coronavirus subtypes. Pediatrics. 2007; 119:e70-e76. [PubMed: 17130280]
5. Rota P A, Oberste M S, Monroe S S, Nix W A, Campagnoli R, Icenogle J P, Penaranda S, Bankamp B, Maher K, Chen M H, Tong S, Tamin A, Lowe L, Frace M, DeRisi J L, Chen Q, Wang D, Erdman D D, Peret T C, Burns C, Ksiazek T G, Rollin P E, Sanchez A, Liffick S, Holloway B, Limor J, McCaustland K, Olsen-Rasmussen M, Fouchier R, Gunther S, Osterhaus A D, Drosten C, Pallansch M A, Anderson L J, Bellini W J. Characterization of a novel coronavirus associated with severe acute respiratory syndrome. Science. 2003; 300:1394-1399. [PubMed: 12730500]
6. Marra M A, Jones S J, Astell C R, Holt R A, Brooks-Wilson A, Butterfield Y S, Khattra J, Asano J K, Barber S A, Chan S Y, Cloutier A, Coughlin S M, Freeman D, Girn N, Griffith O L, Leach S R, Mayo M, McDonald H, Montgomery S B, Pandoh P K, Petrescu A S, Robertson A G, Schein J E, Siddiqui A, Smailus D E, Stott J M, Yang G S, Plummer F, Andonov A, Artsob H, Bastien N, Bernard K, Booth T F, Bowness D, Czub M, Drebot M, Fernando L, Flick R, Garbutt M, Gray M, Grolla A, Jones S, Feldmann H, Meyers A, Kabani A, Li Y, Normand S, Stroher U, Tipples G A, Tyler S, Vogrig R, Ward D, Watson B, Brunham R C, Kraj den M, Petric M, Skowronski D M, Upton C, Roper R L. The Genome sequence of the SARS-associated coronavirus. Science. 2003; 300:1399-1404. [PubMed: 12730501]
7. Kuiken T, Fouchier R A, Schutten M, Rimmelzwaan G F, van Amerongen G, van Riel D, Laman J D, de Jong T, van Doornum G, Lim W, Ling A E, Chan P K, Tam J S, Zambon M C, Gopal R, Drosten C, van der Werf S, Escriou N, Manuguerra J C, Stohr K, Peiris J S, Osterhaus A D. Newly discovered coronavirus as the primary cause of severe acute respiratory syndrome. Lancet. 2003; 362:263-270. [PubMed: 12892955]
8. Zaki A M, van Boheemen S, Bestebroer T M, Osterhaus A D, Fouchier R A. Isolation of a novel coronavirus from a man with pneumonia in Saudi Arabia. The New England journal of medicine. 2012; 367:1814-1820. [PubMed: 23075143]
9. van Boheemen S, de Graaf M, Lauber C, Bestebroer T M, Raj V S, Zaki A M, Osterhaus A D, Haagmans B L, Gorbalenya A E, Snijder E J, Fouchier R A. Genomic characterization of a newly discovered coronavirus associated with acute respiratory distress syndrome in humans. mBio. 2012:3.
10. Perlman S, Dandekar A A. Immunopathogenesis of coronavirus infections: implications for SARS. Nature reviews. Immunology. 2005; 5:917-927.
11. Perlman S, Netland J. Coronaviruses post-SARS: update on replication and pathogenesis. Nature reviews. Microbiology. 2009; 7:439-450.
12. Immunol Res. 2014 August; 59(0): 118-128. doi:10.1007/s12026-014-8534-z
13. PCT/US2005/028778

What is claimed is:

1. A method of making a composition for treating an infectious disease, said method comprising:
a) obtaining a cell line of human pluripotent stem (hPS) cells;
b) differentiating said hPS cells into a population of mature antigen-presenting cells, wherein said antigen expressing cells express an antigen of an infectious disease pathogen;
c) genetically altering said hPS cells before or after they are differentiated so that they express a protein comprising one or more immunogenic epitopes of said infectious disease pathogen; and
d) formulating said differentiated cells to provide a composition for administration to a human subject.

2. The method of claim 1, wherein the infectious disease pathogen is a virus.

3. The method of claim 1 or 2, wherein the virus is a Coronaviridae virus.

4. The method of claim 3, wherein said Coronaviridae virus is SARS-CoV-2.

5. The method of any of claims 1 to 4, wherein said antigen-presenting cells are selected from the group consisting of dendritic cells, macrophages, B cells, and a combination thereof.

6. The method of any one of claims 1-5, wherein said composition is administered to a human for treating a disease caused by a Coronaviridae virus infection.

7. The method of any one of claims 1-5, wherein said composition is administered to a human for preventing a disease caused by a Coronaviridae virus infection.

8. The method of any one of claims 1-7, wherein said composition is a vaccine.

9. The method of any of claims 3 to 8, wherein the Coronaviridae antigen comprises the Coronaviridae spike protein.

10. The method of claim 9, wherein the Coronaviridae antigen comprises the full-length spike protein.

11. The method of claim 9, wherein the Coronaviridae antigen comprises the spike protein receptor-binding domain (RBD).

12. The method of claim 11, wherein the Coronaviridae antigen comprises a 209 amino acid sequence of the RBD.

13. The method of claim 9, wherein the Coronaviridae antigen comprises the spike protein S1 domain, S2 domain, or both.

14. The method of any of claims 3 to 8, wherein the Coronaviridae antigen comprises the nucleocapsid protein.

15. The method of any of claims 3 to 8, wherein the Coronaviridae antigen comprises the membrane protein.

16. The method of any of claims 1 to 15, wherein genetically altering said hPS cells comprises transfecting the cells with a polynucleotide encoding a Coronaviridae virus antigen and the cytoplasmic tail of lysosomal associated membrane protein 1 (LAMP-1).

17. The method of claim 16, wherein the polynucleotide further comprises a poly(A) tail and a gene which encodes heat shock protein 96 (HSP96).

18. A composition according to any one of claims 1-17.

19. The method of claim 18, wherein the infectious disease pathogen is a virus.

20. The method of claim 19, wherein the virus is a Coronaviridae virus.

21. The composition of claim 20, wherein said Coronaviridae virus is SARS-CoV-2.

22. The composition of any of claims 18 to 21, wherein said antigen-presenting cells are selected from the group consisting of dendritic cells, macrophages, B cells, and a combination thereof.

23. The composition of claim 22, wherein said antigen-presenting cells are dendritic cells.

24. The composition of any one of claims 18 to 23, wherein said composition is administered to a human for treating a disease caused by a Coronaviridae virus infection.

25. The composition of any one of claims 18 to 23, wherein said composition is administered to a human for preventing a disease caused by a Coronaviridae virus infection.

26. The composition of any of claims 18 to 25, wherein the Coronaviridae antigen comprises the Coronaviridae spike protein.

27. The composition of claim 26, wherein the Coronaviridae antigen comprises the full-length spike protein.

28. The composition of claim 26, wherein the Coronaviridae antigen comprises the spike protein receptor-binding domain (RBD).

29. The composition of claim 26, wherein the Coronaviridae antigen comprises a 209 amino acid sequence of the RBD.

30. The composition of claim 26, wherein the Coronaviridae antigen comprises the spike protein S1 domain, S2 domain, or both.

31. The composition of any of claims 18 to 25, wherein the Coronaviridae antigen comprises the nucleocapsid protein.

32. The composition of any of claims 18 to 25, wherein the Coronaviridae antigen comprises the membrane protein.

33. The composition of any of claims 18 to 32, wherein genetically altering said hPS cells comprises transfecting the cells with a polynucleotide encoding a Coronaviridae virus antigen and the cytoplasmic tail of lysosomal associated membrane protein 1 (LAMP-1).

34. The method of claim 33, wherein the polynucleotide further comprises a poly(A) tail and a gene which encodes heat shock protein 96 (HSP96).

35. The composition of any one of claims 18-34, wherein said composition is a vaccine.

36. A composition comprising dendritic cells expressing a Coronaviridae antigen.

37. The composition of claim 36, wherein the Coronaviridae antigen comprises the Coronaviridae spike protein.

38. The composition of claim 37, wherein the Coronaviridae antigen comprises the full-length spike protein.

39. The composition of claim 37, wherein the Coronaviridae antigen comprises the spike protein receptor-binding domain (RBD).

40. The composition of claim 39, wherein the Coronaviridae antigen comprises a 209 amino acid sequence of the RBD.

41. The composition of claim 37, wherein the Coronaviridae antigen comprises the spike protein S1 domain, S2 domain, or both.

42. The composition of claim 36, wherein the Coronaviridae antigen comprises the Coronaviridae nucleocapsid protein.

43. The composition of claim 36, wherein the Coronaviridae antigen comprises the Coronaviridae membrane protein.

44. The composition of any of claims 36 to 43, wherein genetically altering said hPS cells comprises transfecting the cells with a polynucleotide encoding a Coronaviridae virus antigen and the cytoplasmic tail of lysosomal associated membrane protein 1 (LAMP-1).

45. The method of claim 44, wherein the polynucleotide further comprises a poly(A) tail and a gene which encodes heat shock protein 96 (HSP96).
